(19) United States
(12) Patent Application Publication
(10) Pub. No.: US 2008/0132440 A1
(43) Pub. Date: Jun. 5, 2008

(54) Poly-Gamma-Glutamic Acid-Vitamin Complex and Use Thereof

(76) Inventors: Moon-Hee Sung, Daejeon (KR); Chung Park, Daejeon (KR); Seok Chan Kim, Gyeonggi-do (KR); Gyoo Soon Park, Gyeonggi-do (KR); Hiroshi Uyama, Shiga (JP); Ha Ryoung Poo, Daejeon (KR); Jae Jun Song, Daejeon (KR)

Correspondence Address: INTELLECTUAL PROPERTY / TECHNOLOGY LAW, PO BOX 14329, RESEARCH TRIANGLE PARK, NC, 27709, US

(21) Appl. No.: 11/571,269

(22) PCT Filed: Mar. 4, 2005
(86) PCT No.: PCT/KR05/00603

§ 371 Date: Jan. 15, 2007

(30) Foreign Application Priority Data
Jun. 24, 2004 (KR) 10-2004-0047863

(51) Int. Cl. A61K 38/02; A61P 17/00; A61P 3/02; C07K 2/00; A61Q 1/00

(52) U.S. Cl. 514/2; 530/345

(57) ABSTRACT

The present invention relates to PGA-vitamin complex containing poly-γ-glutamic acid(PGA), vitamin preparations containing said PGA-vitamin complex or cosmetic compositions. The inventive PGA-vitamin complex having excellent hygroscopicity, moisturizing property and skin compatibility, which is a complex of poly-γ-glutamic acid and vitamin, has the effect of sustained-release as well as improves stability of vitamin having the functions, such as promotion of metabolism, anti-oxidation effect, protection of cell wall, increasing immunity, prevention of dry skin and keratinization, anti-wrinkles and moisturizing skin, thereby being useful as cosmetic compositions and sustained-release vitamin preparations for various applications.

### TECHNICAL FIELD

This invention relates to a PGA-vitamin complex containing PGA (poly-gamma-glutamic acid), and a vitamin preparation or a cosmetic composition containing the PGA-vitamin complex.

### BACKGROUND ART

Vitamins are regarded as the most representative cosmetic ingredients, even a tiny amount of which normalize the physiological and metabolic functions of skin and prevent skin diseases caused by vitamin deficiency. Vitamins having functions of promoting metabolism, anti-oxidation effect, protecting cell wall, increasing immunity, strengthening resistance to infection, and the like are essential substances in the body and biosynthesis of vitamins is impossible, so that food is the only way for one's vitamin intake, and also lack of vitamin causes various symptoms of deficiency. Furthermore, in terms of beautifying and curing skin, vitamins play an important role in maintaining healthy skin by preventing pigmentation, promoting collagen synthesis, protecting of ultraviolet rays, preventing keratinization and dry skin, anti-wrinkles, and moisturizing skin.

Vitamins include retinol (Vit. A), ascorbic acid (Vit. C), tocopherol (Vit. E), vitamin D and derivatives thereof. Vitamin C called ascorbic acid is one of essential nutrients, which is impossible to be synthesized in the body. Vitamin C is a basic substance for collagen formation, so that is needed for repair and growth of tissue and also it is an essential ingredient for repairing a fracture as well as for strengthening the gums, improving the function of renal capsule and facilitating the absorption of iron. Furthermore, as vitamin C has anti-oxidation function, it prevents oxidation by returning oxidative substance to reductive substance in the body. Since vitamin C itself oxidizes easily to be dissolved and has difficulty being absorbed into skin due to its water-soluble property, ester-type vitamin C palmitate is mainly used to increase its stability and to promote the absorption of vitamin C into skin.

Vitamin D is generally known as a compound used for the prevention of rickets and a cure for rickets and mainly includes ergocalciferol (Vit. D₂) and cholecalciferol (Vit. D₃). Ergocalciferol derived from plant-steroid ergosterol is generally used as a vitamin D-enriching agent in food. Ring-opening seco-steroid type, cholecalciferol, which is a vitamin D generated in skin, is generated from 7-dehydrocholesterol by radiation and the name cholecalciferol shows that it is related to cholesterol and calcium. As described above, Vitamin D can not be regarded as a typical vitamin since it is synthesized in the body and can be classified as prohormone due to its change to physiologically active substance like steroid hormone in the process of metabolism. Typically known main functions of vitamin D are to participate in the absorption of calcium and phosphate and the formation of bond along with calcitonin and PTH (parathyroid hormone). Recently, functions regarding reproduction, immune effect and gene regulation, and the like, have been drawing attention.

Kojic acid having the function of whitening skin, indoleacetic acid having the function of anti-wrinkles, lactic acid, citric acid and salicylic acid helping the removal of stratum corneum and metabolism as well as vitamins are known as significant cosmetic ingredients.

However, there are a number of difficulties and limitations in the methods for usage thereof, which most of cosmetic ingredients are not fully functioning and effecting due to the problems such as instability, skin irritation, sustained release, dispersiveness and toxicity of ingredients per se. In the case of vitamins, it is very unstable physicochemically and easily broken down by heat, light, humidity, oxygen, alkali, etc, and thus is discolored, malodorous or its function and effect is lessened. Therefore a lot of studies on technical development for formulation to stabilize cosmetic ingredients and reduce skin irritations and toxicity are being reported (KR 2000-0048451, 2000-0069893 and 1999-0070885).

PGA is a polymer which D, L-glutamic acid is bound to γ-glutamyl, and is a mucous substance. PGA is produced from the genus Bacillus strain isolated from traditional fermented soybean food such as chungkookjang in Korea, natto in Japan or kinema in Nepal using straw. PGA produced from the genus Bacillus strain is a macromolecular substance which is edible, water-soluble, negative ionic and biodegradable and it is possible to use PGA for a moisturizer, hygroscopic agent, and cosmetic ingredients. Lately, studies on alternative goods materials of non-dissolvable polymer, the development of heat-resistant plastics by esterification, and the production of water-soluble fibers and membranes in relation to studies on the production and usage of PGA are actively being conducted centering around developed countries.

Moreover, studies on changes in property caused by irradiating γ-rays to PGA, and the development and industrialization of hydrogel by cross-linker are being promoted. Hydrogel is a material characterized by absorptivity, biodegradability and plasticity, which is synthesized by cross-linkage between molecules or same molecules using PGA which is fermented and produced by Bacillus subtillis var choongkookjang and is a biopolymer possible to be reproduced, as an ingredient. Methods for cross-linkage include, such as irradiation of radioactive rays of γ-ray and e-ray, and so on and chemical cross-linker treatment with epoxy resin, and so on. Upon irradiating radioactive rays to a water solution, cross-linkage reaction occurs between PGA molecules whereby PGA resin characterized by absorptivity, biodegradability and plasticity is obtained.

Studies on PGA formulation, the effect of manganese ion on PGA production, use of PGA as a water-soluble polymer by ultrasonic dissolution and the development of plastics with low water-solubility by synthesis of ester derivative (Biosci,. Biotechnol. Biochem. 60(8):1239-1242, 1996), the production of PGA by Bacillus subtillis and the application of PGA to health food for a cure for osteoporosos as a calcium dissolvent (JP 6-32742) were reported.

Besides, there are reports on effect of decreasing water pollution by reducing phosphorus content in sewage (EP 838160) and the application (JP 10-251402) and utilization (JP 7-300522 and 6-322358) in food, horticultural industry and sanitary supplies such as a diaper by preparing biodegradable resin having high gelatinization, absorptivity and adsorptive property by irradiating radioactive rays to PGA. Also, use as solidified biodegradable fibers, films and film-forming composition by dissolution and precipitation of PGA and then drying (JP 7-138364 and 5-117388), and a polymer for a drug carrier (JP 6-92870 and 6-256220) were reported.

On the other hand, in Korea, basic studies, such as an effective production of PGA (KR 1997-0003404 and 1997-0067605) and improvement in the property of matter, the method of producing PGA with high concentration (KR 2001-0106025), and a salt-tolerant Bacillus subtillis var. chungkookjang strain producing PGA with high molecular weight (KR 2001-0001481) were reported.

Accordingly, the present inventors have made extensive efforts to develop a substance to improve stability of vitamins and derivatives thereof used widely in the field of medicine and cosmetics, and consequently prepared PGA-vitamin complex having hygroscopicity, moisturizing property and skin compatibility by remarkably improving problems, such as instability of vitamins, skin irritations, toxity and sustained-release, thereby perfecting this present invention.

### DISCLOSURE OF INVENTION

It is an object of the present invention to provide a PGA-vitamin complex and a method for preparing the same.

Another object of the present invention is to provide a vitamin preparation and a cosmetic composition containing the PGA-vitamin complex as an effective ingredient.

To accomplish the above object, in one aspect, the present invention provides the PGA-vitamin complex having hydroxyl group of vitamin linked with carboxyl group of PGA by ester bond.

In the present invention, said vitamin include water-soluble vitamin C, fat-soluble vitamin D, or derivatives thereof. Derivatives of water-soluble vitamin C include ethylascorbyl ether, magnesium ascorbyl phosphate, ascrobic acid 2-glucoside and allantoin ascorbate, and derivatives of fat-soluble vitamin D include ergocalciferol (Vit. D₂) and cholecalciferol (Vit. D₃).

In another aspect, the present invention provides PGA-water-soluble vitamin C complex represented by the following formula I:

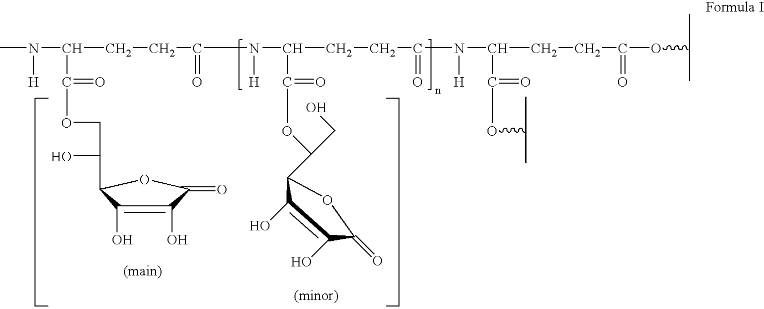

In still another aspect, the present invention provides PGA-fat-soluble vitamin D (or derivative thereof) complex represented by the following formula II:

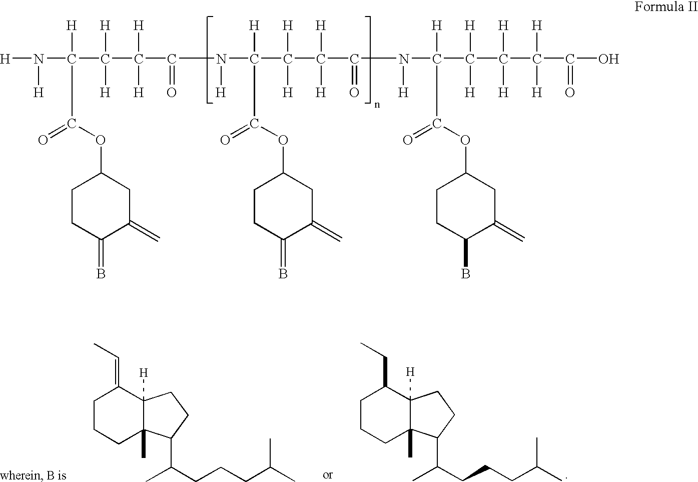

In yet another aspect, the present invention provides PGA-fat-soluble vitamin D (or derivative thereof) complex represented by the following formula III characterized by coupling of PGA and fat-soluble-vitamin D (or derivative thereof) by a linker:

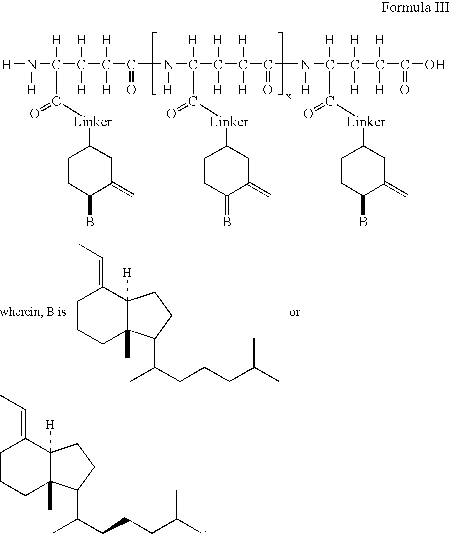

In the present invention, the linker is H₂N—R₁—NH₂, H₂N—R₂—SH, H₂N—R₃—OH, H₂N—R₄—CHO, HS—R₅—SH, or HO—R₆—OH. Wherein, R₁, R₂, R₃, R₄, R₅ and R₆ is C₁₋₂₀ saturated hydrocarbon, unsaturated hydrocarbon or aromatic organic group, respectively.

In still another aspect, the present invention provides a method for preparing PGA-vitamin complex, the method comprises linking carboxyl group of PGA with hydroxyl group of vitamin by ester bond.

In the present invention, the PGA-vitamin complex is prepared by Bacillus subtilis.

In yet another aspect, the present invention provides a vitamin preparation and food containing the PGA-vitamin complex as an effective ingredient.

In a further aspect, the present invention provides beverage containing the PGA-vitamin C complex as an effective ingredient.

In further another aspect, the present invention provides a cosmetic composition for improvement of skin compatibility, moistening property and/or hygroscopicity, containing the PGA-vitamin complex as an effective ingredient.

In composition of the present invention, the weight of PGA-vitamin complex is 0.01 to 60 wt %, preferably 0.1 to 50 wt % based on the total weight of the composition.

Cosmetic compositions of the present invention can be combined with substances, mixed with typical cosmetic composition, e.g. oil, water, surfactant, moisturizer, low quality alcohol, thickener, chelating agent, pigment, antiseptic, perfume, etc. as much as needed.

Cosmetic compositions containing vitamin complex with PGA of the present invention can be variously applied to moisturizer, cleanser, a body lotion etc.

Products which the inventive compositions can be added are cosmetics, such as an astringent lotion, a moisturizer, a nourishing lotion, various creams, essence, pack, foundation and cleanser, soap, conditioner, cosmetic liquid, and so on.

Examples of cosmetic composition of the present invention, there are a moisturizer, a skin softener, a toner, an astringent, a lotion, an emulsion, a moisturizing lotion, a nourishing lotion, a massage cream, a nourishing cream, a moisturizing cream, a cream for hand, essence, nourishing essence, pack, soap, shampoo, cleansing foam, a cleansing lotion, cleansing cream, a body lotion, body cleanser, oil for body, pressed powder, loose powder and eye shadow, and so on.

### BRIEF DESCRIPTION OF DRAWINGS

FIG. 1 shows the formula of vitamin D₂ (erogocalciferol) chemical structure.

FIG. 2 shows the formula of vitamin D₃ (cholecalciferol) chemical structure.

### DETAILED DESCRIPTION OF THE INVENTION

Hereinafter, the present invention will be described in further detail by examples. It will however be obvious to a person skilled in the art that these examples are given for illustrative purpose only, and the scope of the present invention is not limited to or by these examples.

### EXAMPLE 1

### Preparation of PGA(poly-γ-glutamic Acid)

After 1% culture broth of Bacillus subtilis var chungkookjang (KCTC 0697BP) strain was inoculated in 5 L incubator having 3 L of basic culture media used for PAG production (5% L-glutamic acid added to GS culture medium: 5% glucose, 1% (NH₄)₂SO₄, 0.27% KH₂PO₄, 0.42% Na₂HPO₄.12H₂O, 0.05% NaCl, 0.3% MgSO₄.7H₂O, 1 ml/L of vitamin solution, pH 6.8), cultured at the stirring rate of 150 rpm and the air inflow rate of 1 vvm at 37° C. for 72 hours, and then 2N sulfate solution was added adjusting to pH 3.0 to obtain sample solution containing PGA.

The sample solution containing PGA was left as it was at 4° C. for 10 hours to remove polysaccharide in fermentation solution, and then fully mixed with ethanol two times the volume of fermentation solution. After the mixture was left as it was at 4° C. for 10 hours, precipitated PGA was obtained by centrifugation. Distilled water was added to the obtained precipitate to dissolve and protease was added to be 100 μg/ml, and then left as it was in a 37° C. thermostatic incubator for 6 hours to dissolve extracelluar protein in PGA sample. The separated glutamic acid was removed by dialysis in sufficient amount of distilled water, and then concentrated whereby pure PGA was obtained.

### EXAMPLE 2

### Preparing a Comlex of PGA and Water-Soluble Vitamin C

0.65 g of PGA produced in Example 1 was dissolved in 6.5 ml DMSO under the condition of argon (or nitrogen) in a dry test tube. Transparent liquid is obtained within several hours by stirring the mixture at room temperature, and then 0.58 g of 1-ethyl-3-(3-dimethylaminopropyl) carbodiimide hydrochloride and 0.23 g N-hydroxysuccinimide which is an activating agent were added to the obtained transparent liquid, followed by stirring at room temperature. 6 hours later, gel was formed by adding 0.03 g of water-soluble vitamin C dissolved in 1 ml DMSO, and then the formed gel was soaked in sufficient water. After exchanging the water several times, it was freeze-dried whereby a complex of water-soluble and powdery PGA and water-soluble vitamin C was obtained. The reaction of PGA and water-soluble vitamin C is represented by the reaction formula 1 as follows.

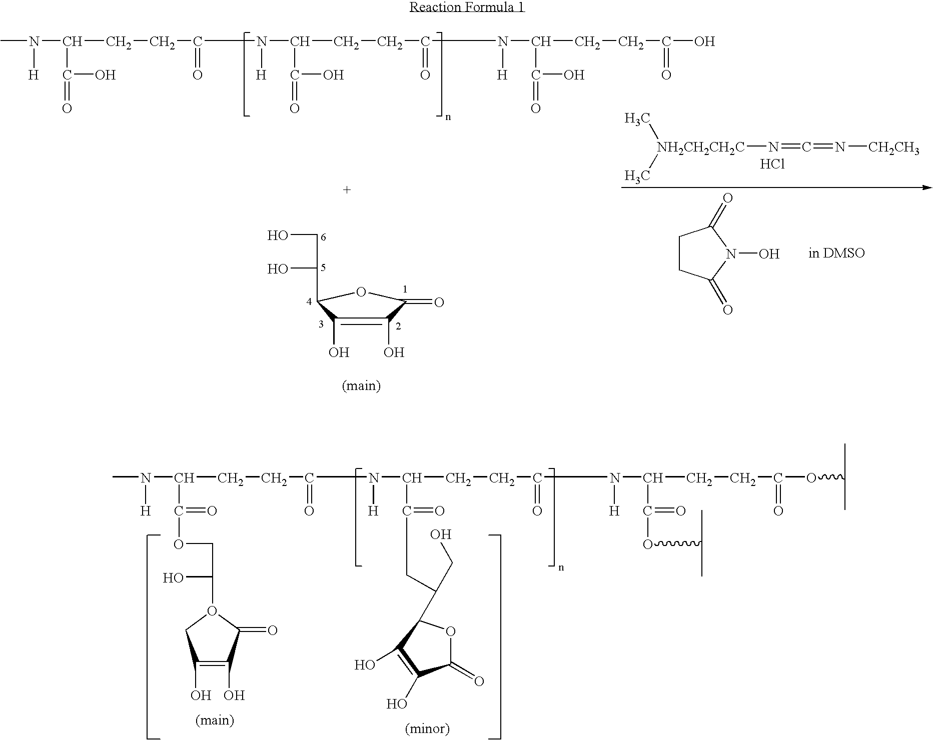

By performing the same method as the above, a complex of PGA and vitamin C derivative, such as ethylascorbyl ether, magnesiumascorbyl phosphate ascrobic acid 2-glucoside or allantoin ascorbate can be prepared.

### EXAMPLE 3

### Preparing a Complex of PGA and Fat-Soluble Vitamin D

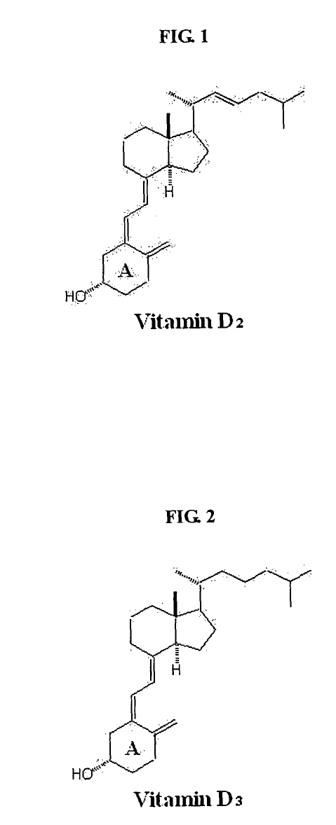

In comparison with typical steroid compounds, fat-soluble vitamin D has similarities in structure, however vitamin D has conjugated triene bond by splitting off of 9^{th} and 10^{th} carbon bonds unlike typical steroid compounds. Also, double bonds of 10^{th}-19^{th} carbons are distorted at 60° from a horizontal plane. As a result, A-ring can exist as two possible forms of chair structure, from which rigid CD-ring and side chain are extended. The conformational mobility is not found in other steroid hormones except fat-soluble vitamin D molecule which is seco-steroid. Since A-ring becomes “free” by splitting off of 9^{th} and 10^{th} carbon bonds, substitutive group of A-ring can be changed to axial and equatorial positions (FIG. 1 and FIG. 2). In connection with that, the properties of vitamin D are different from those of other steroids, such as vitamin D receptor being inconsistent with other steroid receptors, which is explained to be caused by conformational mobility of A-ring, which is an unique characteristic of seco-B steroid unlike other steroids with lack of mobility of rings. As represented by the following reaction formula 2, PGA produced in the Example 1 and A-ring of fat-soluble vitamin D was subjected to coupling reaction using DMSO solvent, 1-ethyl-3-(3-dimethylaminopropyl) carbodiimide hydrochloride and N-hydroxysuccinimide to obtain a complex of PGA and fat-soluble vitamin D (or derivative thereof). Ethylenediamine or ethylene glycol was used as a linker.

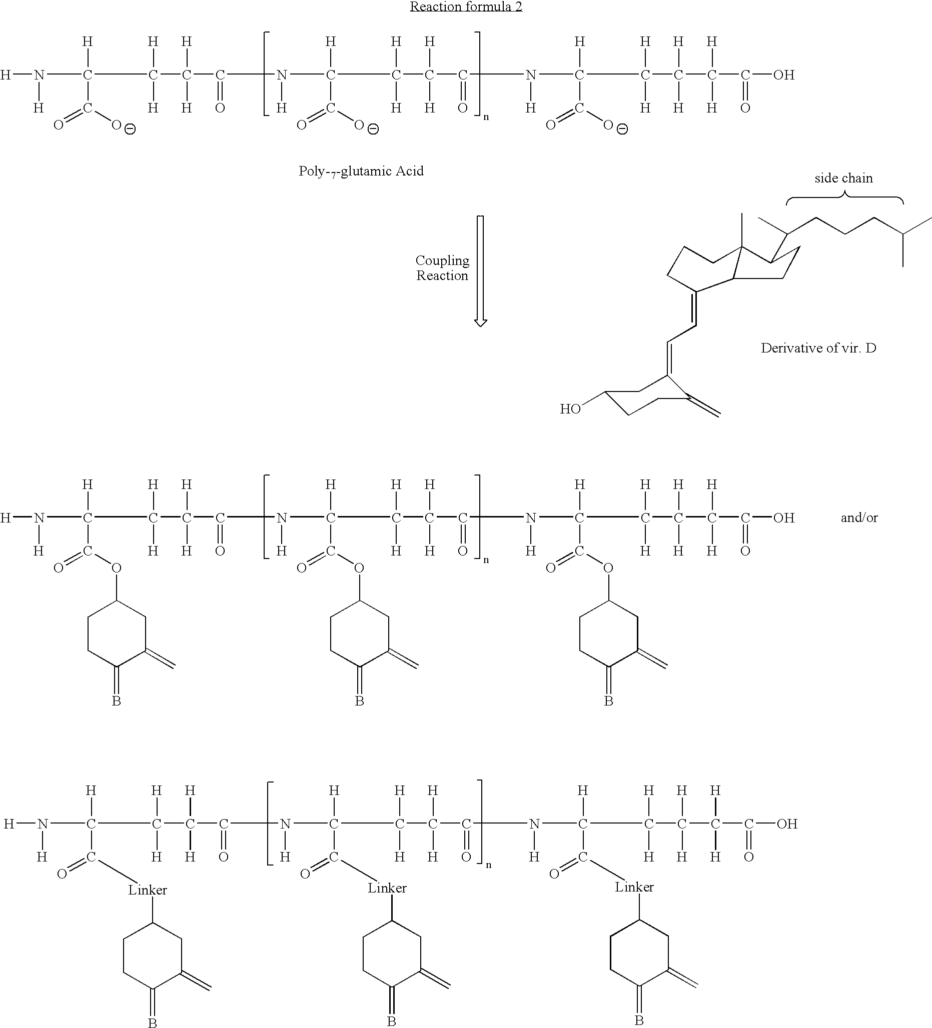

### EXAMPLE 4

### Sustained-Release Effect of PGA-Vitamin Complex

An experiment was performed to determine whether PGA-vitamin complex prepared in Examples 2 and 3 has sustained-release effect in intestinal absorption thereof according to the method in KR 2003-0046898 by the present inventors as follows.

That is, thirty 4-week old Balb/c male mice were bred in a mouse cage where optimum temperature and the cycle of 12 hour light and 12 hour darkness are controlled, and they were provided with basic feed and distilled water. 1 hour, 1.5 hours and 2 hours after oral administration of PGA-vitamin complex, the mice were etherized and then the whole small intestines from duodena to ilea was taken off from abdomina of the mice. The separated small intestines was divided into upside and downside and washed with cold physiological salt solution, followed by homogenizing the small intestinal tissues by homogenizer, adding proper amount of cold physiological salt solution. After the homogenized small intestinal tissues were centrifuged at 8,000 g, 4° C. for 20 minutes, vitamins contained were analyzed with HPLC while the separated soluble parts and insoluble precipitates of each fragment were stored at −20° C.

As a result, it was confirmed that PGA-vitamin complex of the present invention has significant sustained-release as the intestinal absorption rate of vitamin was increased with the passage of time.

### EXAMPLE 5

### Effect of Improving Stability of Vitamin of PGA-Vitamin Complex

To test the effect of improving vitamin stability in the cosmetic formulation of PGA-vitamin complex obtained by the Examples 2 and 3, an experiment was performed as follows.

### 5-1: Stability Test According to Changes in Temperature

A lotion containing PGA-vitamin complex formulated in the following Example of Formulation 2 as a sample group and a lotion containing only vitamin instead of PGA-vitamin complex as a control group put in a opaque glass vessel were stored in a 45° C. thermostatic incubator for 1 week. Also, the sample group and control group in a opaque glass vessel were stored in a 4° C. refrigerator without light for 1 week. 1 week after the storage, degrees of discoloration was compared and measured (evaluation standards—0: no change, 1: very little changed, 2: a little changed, 3: less badly changed, 4: badly changed, 5: very badly changed).

As a result, a lotion containing PGA-vitamin complex was hardly discolored, so it was confirmed that PAG-vitamin complex is very effective to stabilize vitamin (Table 1).

TABLE 1

Degrees of discoloration

| Temp. | Sample group | Control group |
|---|---|---|
| 45° C. | 2.5 | 0.5 |
| 4° C. | 1.0 | 1.0 |

### 5-2: Stability Test with the Passage of Time

A lotion containing PGA-vitamin complex formulated by the following Example of Formulation 2 as a sample group and a lotion containing only vitamin instead of PGA-vitamin complex as a control group put in the opaque glass vessel were stored in a 20° C. thermostatic incubator for 9 weeks to measure the amount of vitamin. As a result, the vitamin content of a lotion containing inventive PGA-vitamin complex was hardly changed, so it was confirmed that PAG-vitamin complex is very effective to stabilize vitamin (Table 2).

TABLE 2

| | 0 week | 3 weeks | 6 weeks | 9 weeks |
|---|---|---|---|---|
| Control group | 40,010 μg/g | 33,420 μg/g | 19,050 μg/g | 10,991 μg/g |
| Sample group | 40,080 μg/g | 38,899 μg/g | 35,180 μg/g | 28,670 μg/g |

### EXAMPLE 6

### Measuring Moisturizing Ability of PGA-Vitamin Complex

The moisturizing ability of essence containing PGA-vitamin complex formulated in the following Formulation Example 3 was compared to that of essence without PGA-vitamin complex in the following comparative Formulation Example 3. Electrical conductivity on skin surface was measured for measurement of the moisturizing ability using Corneometer (GmbII, Germany). After applying 0.05 g of each sample to each 16 cm² of skin in 25° C. thermostatic room with 40% constant relative humidity, the amount of moisture loss was measured 30 minutes and 2 hours after the application. Experiments for measurements were performed three times and the number of subjects was 20.

As a result, it was confirmed that the moisture-maintaining ability of the composition containing inventive PGA-vitamin complex is superior to that of composition without PGA-vitamin complex (Table 3).

TABLE 3

| | Cosmetic composition with | Cosmetic composition without |
|---|---|---|
| | PGA-vitamin complex | PGA-vitamin complex |
| 30 mins after | 125 | 105 |
| application | | |
| 2 hrs after | 102 | 70 |
| application | | |

The average value was about 60 before application of sample and the values shown was obtained after averaging the measured values of 3 experiments.

### EXAMPLE 7

### Safety Test on Skin

Safety of cosmetic composition containing PGA-vitamin complex on skin was tested. Specifically, 30 subjects (The average age was 25, people aged between 19 and 40) were divided into A and B groups, and A group with the inventive cosmetic composition and B group with the cosmetic composition formulated in comparative formulation Example were subjected to Patch Test using Haye's Test Chamber.

At this time, people with the skin diseased symptoms, such as psoriasis or eczema, pregnant women, nursing mothers or people taking antihistamines were eliminated. After test sites were washed with 70% ethanol, and dried, 15 μg of each sample dropped down into chambers was fixed on the upper arms of each A group and B group. Patch was applied to the test sites for 24 hours and removed, and then the test sites were marked with marking pen, followed by observing the test sites after 24 hours, 48 hours and 72 hours. The test results were determined according to regulations of International Contact Dermatitis Research Group (ICDRG) (see Table 4).

TABLE 4

| Symbol | Criteria | Evaluation | Average |
|---|---|---|---|
| ± | doubtful response or | little irritations | 0-0.9 |
| | little response and erythema | | |
| + | erythema + cirrhosis | mild irritations | 1.0-2.9 |
| ++ | erythema + cirrhosis + vesicle | less strong irritations | 3.0-4.9 |
| +++ | erythema + cirrhosis + vesicle | strong irritations | ≧5.0 |
| − | no response | no irritations | 0 |

As a result, it was found that the composition containing PGA-vitamin complex of the present invention is safe cosmetic composition without skin irritations, resulting in the average stimulus level of 0 whereby it was confirmed that PGA-vitamin complex of the present invention has high skin compatibility (see Table 5).

TABLE 5

| | Cosmetic composition with | Cosmetic composition without |
|---|---|---|
| Time (hr) | PGA-vitamin complex | PGA-vitamin complex |
| 24 | 0 | 0.5 |
| 48 | 0 | 0.5 |
| 72 | 0 | 0 |

Hereinafter, as the formulation Examples of the present invention, moisturizer, a lotion, essence, cleanser (cleansing foam) and shampoo are exemplified, however, the formulation containing cosmetic composition of the present invention is not restricted by the Examples.

### Example of Formulation 1: Moisturizer (Skin)

After butylene glycol, glycerine, polyoxyethylene (60) hydrogenated castor oil, betaine, citric acid, sodium citrate and antiseptic were added to purified water, stirred, and dissolved, perfume dissolved in ethanol was added. PGA-containing vitamin complex was added to the mixture and fully stirred, and then ripened whereby moisturizer containing PGA-vitamin complex was prepared. The content of each ingredient is shown in the following Table 6.

TABLE 6

| | Formulation | Comparative |
|---|---|---|
| | Example 1 | formulation |
| Ingredient | (w/w %) | Example 1 (w/w %) |
| PGA-vitamin complex | 50.0 | — |
| Butylene glycol | 7.0 | 7.0 |
| Glycerine | 5.0 | 5.0 |
| Polyoxyethylene (60) | 0.2 | 0.2 |
| hydrogenated castor oil | | |
| Ethanol | 5.0 | 5.0 |
| Betaine | 2.0 | 2.0 |
| Citric acid | 0.02 | 0.02 |
| Sodium citrate | 0.06 | 0.06 |
| Antiseptic | small amount | small amount |
| Perfume | small amount | small amount |
| Purified water | residual | residual |

### Example of Formulation 2: Lotion (Emulsion)

After PGA-containing vitamin complex prepared in the above Example, butylene glycol, glycerine, carboxyvinylpolymer, arginie, antiseptic and purified water were heated at 70° C.˜75° C. while being stirred. A mixture of squalane, butylene glycol dicaprylate/dicaprate, sorbitan stearate, polysorbate 60, glyceryl stearate and stearyl glyceratinate, obtained by stirring and heating at 75° C.˜80° C., was added to the former mixture to emulsify. The mixture was stirred to cool until it reaches about 45° C., and then perfume was added and stirred. Finally the mixture was cooled to 30° C., and then ripened whereby lotion containing PGA-vitamin complex was prepared. The content of each ingredient is shown in the following Table 7.

TABLE 7

| | Formulation | Comparative |
|---|---|---|
| | Example 2 | formulation |
| Ingredient | (w/w %) | Example 2 (w/w %) |
| PGA-vitamin complex | 40.0 | — |
| Butylene glycol | 8.0 | 8.0 |
| Glycerine | 5.0 | 5.0 |
| Squalane | 10.0 | 10.0 |
| Butylene glycol | 5.0 | 5.0 |
| dicaprylate/dicaprate | | |
| Sorbitan stearate | 1.5 | 1.5 |
| Polysorbate 60 | 1.0 | 1.0 |
| Glyceryl stearate | 0.5 | 0.5 |
| Stearyl glyceratinate | 0.2 | 0.2 |
| Carboxyvinyl polymer | 0.1 | 0.1 |
| Arginine | 0.1 | 0.1 |
| Antiseptic | small amount | small amount |
| Perfume | small amount | small amount |
| Purified water | residual | residual |

### Example of Formulation 3: Essence

After sito sterol, polyglyceryl 2-oleate, ceramide, ceteareth-4 and cholesterol were mixed by stirring, a mixture of PGA-vitamin complex, deacetylphosphate, concertrated glycerin and purified water was added, followed by emulsification. The mixture was cooled to 45° C. with stirring and perfume was added and stirred, and then cooled to 30° C., followed by ripening. Carboxyvinyl polymer, xanthan gum and antiseptic were added to the mixture to stabilize and ripened, whereby essence containing PGA-vitamin complex was prepared. The content of each ingredient is shown in the following Table 8.

TABLE 8

| | Formulation | Comparative |
|---|---|---|
| | Example 3 | formulation |
| Ingredient | (w/w %) | Example 3 (w/w %) |
| PGA-vitamin complex | 10.0 | — |
| Sito sterol | 1.70 | 1.70 |
| Polyglyceryl 2-oleate | 1.50 | 1.50 |
| Ceramide | 0.7 | 0.7 |
| Ceteareth-4 | 1.2 | 1.2 |
| Cholesterol | 1.5 | 1.5 |
| Deacetylphosphate | 0.4 | 0.4 |
| Concertrated glycerin | 5.0 | 5.0 |
| Carboxylvinyl polymer | 0.2 | 0.2 |
| Xanthan gum | 0.2 | 0.2 |
| Antiseptic | residual | residual |
| Perfume | residual | residual |
| Purified water | residual | residual |

### Example of Formulation 4: Cleanser (Cleansing Foam)

After N-sodium acylglutamate, glycerine, PEG-400 and propylene glycol were added and mixed to purified water, small amount of PGA-vitamin complex was added, and then EDTA-4Na was added, followed by heating at 80° C. with stirring to dissolve. After a mixture solution of POE (15) oleyl alcohol ether, lauryl derivative and methyl paraben heated at 80° C. was added to the mixture and stirred, perfume was added, and then cooled slowly whereby cleanser containing PGA-vitamin complex was prepared. The content of each ingredient is shown in the following Table 9.

TABLE 9

| | Formulation | Comparative |
|---|---|---|
| | Example 4 | formulation |
| Ingredient | (w/w %) | Example 4 (w/w %) |
| PGA-vitamin complex | 20.0 | — |
| N-sodium acylglutamate | 20.0 | 20.0 |
| Glycerine | 10.0 | 10.0 |
| PEG-400 | 15.0 | 15.0 |
| Propylene glycol | 10.0 | 10.0 |
| POE(15) oleyl alcohol | 3.0 | 3.0 |
| ether | | |
| Lauryl derivative | 2.0 | 2.0 |
| Methyl paraben | 0.2 | 0.2 |
| EDTA-4Na | 0.03 | 0.03 |
| Perfume | 0.2 | 0.2 |
| Purified water | residual | residual |

### Example of Formulation 5: Shampoo

After glycerine and EDTA-4Na were added to purified water, and heated at 80° C. to dissolve, TEA lauryl surfate, sodium laurylether surfate, lauryl amidopropyl betaine and lauric acid diethanol amide were added and stirred. Citric acid was added to the mixture and neutralized at 50° C., and then PGA-vitamin complex and zinc pyrithione were added and stirred at 45° C. whereby shampoo containing PGA-vitamin complex was prepared. The content of each ingredient is shown in the following Table 10.

TABLE 10

| | Formulation | Comparative |
|---|---|---|
| | Example 5 | formulation |
| Ingredient | (w/w %) | Example 5 (w/w %) |
| PGA-vitamin complex | 20.0 | — |
| TEA lauryl surfate | 20.0 | 20.0 |
| Sodium laurylether surfate | 30.0 | 30.0 |
| Lauryl amidopropyl betaine | 2.0 | 2.0 |
| Laurie acid diethanol amide | 3.0 | 3.0 |
| Glycerine | 3.0 | 52.0 |
| EDTA-2Na | 0.05 | 0.05 |
| Methyl paraben | 0.2 | 0.2 |
| Citric acid | 0.03 | 0.03 |
| Zinc pyrithione | 0.02 | 0.02 |
| Perfume | 0.2 | 0.2 |
| Purified water | residual | residual |

While the present invention has been described with reference to the particular illustrative embodiment, it is not to be restricted by the embodiment but only by the appended claims. Accordingly, it is to be appreciated that those skilled in the art can change or modify the embodiment without departing from the scope and spirit of the present invention.

### INDUSTRIAL APPLICABILITY

As described above in detail, the present invention provides PGA-vitamin complex having the effect of increasing stability, promoting the absorption and improving sustained-release of vitamins as well as hygroscopicity, moisturizing property and skin compatibility, and the method for preparing the same. Also the present invention provides the vitamin preparation and the cosmetic composition containing the PGA-vitamin complex as an effective ingredient.

The PGA-vitamin complex according to the present invention has sustained-release effect as well as improving stability and absorption of vitamin having various functions, such as promotion of metabolism, anti-oxidation effect, cell wall protection, increasing immunity, prevention of keratinization and dry skin, anti-wrinkles, and moisturizing skin, thereby being useful as cosmetic compositions and vitamin preparations for various applications.

1. A PGA-vitamin complex, wherein carboxyl group of PGA(poly-gamma-glutamic acid) is linked with hydroxyl group of water-soluble vitamin C, fat-soluble vitamin D, or a derivative thereof by ester bond.

2. The PGA-vitamin complex according to claim 1, wherein the derivative of water-soluble vitamin C is ethylascorbyl ether, magnesium ascorbyl phosphate, ascrobic acid 2-glucoside, or allantoin ascorbate.

3. The PGA-vitamin complex according to claim 1, wherein PGA-water-soluble vitamin C is represented by the following formula I:

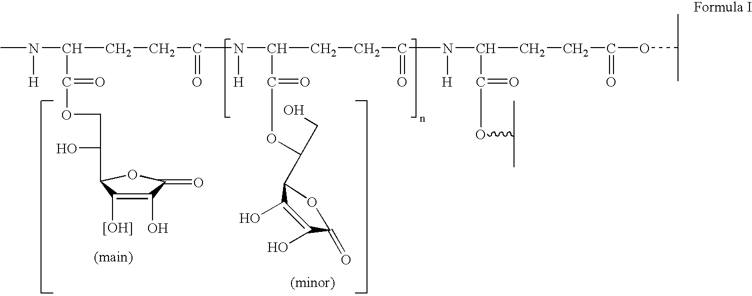

4. The PGA-vitamin complex according to claim 1, wherein the derivative of fat-soluble vitamin D is ergocalciferol (Vit. D₂) or cholecalciferol (Vit. D₃).

5. The PGA-vitamin complex according to claim 1, wherein PGA-fat-soluble vitamin D (or derivative thereof) is represented by the following formula II:

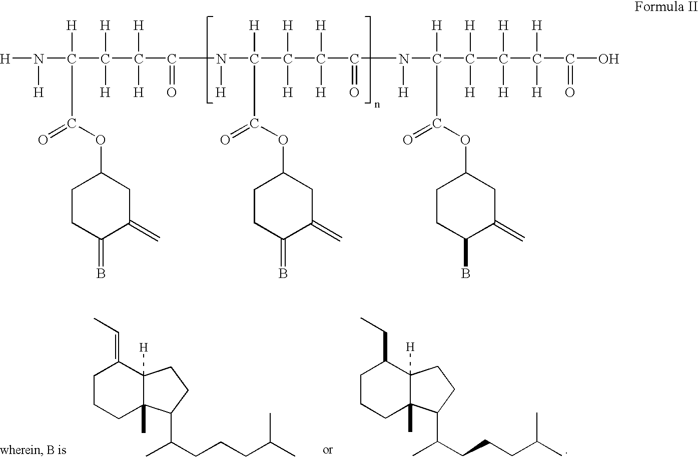

6. The PGA-vitamin complex according to claim 1, wherein PGA is linked with fat-soluble vitamin D (or a derivative thereof) by a linker selected from the group consisting of H₂N—R₁—NH₂, H₂N—R₂—SH, H₂N—R₃—OH, H₂N—R₄—CHO, HS—R₅—SH or HO—R₆—OH (wherein, each R₁, R₂, R₃, R₄, R₅ and R₆ is C₁₋₂₀ saturated hydrocarbon, unsaturated hydrocarbon or aromatic organic group, respectively, and the linked PGA-fat-soluble vitamin D (or a derivative thereof) is represented by the following formula III:

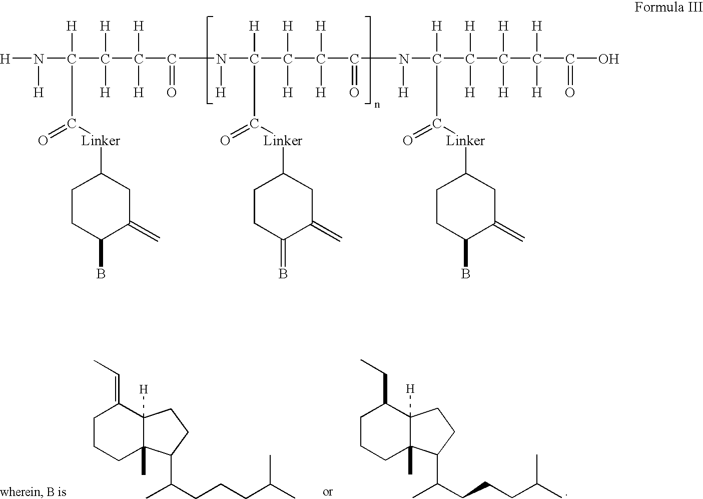

7. The PGA vitamin complex according to claim 1, wherein the PGA is prepared by Bacillus subtilis.

8. A method for preparing the PGA-vitamin complex, comprising linking carboxyl group of PGA with hydroxyl group of vitamin by ester bond.

9. A vitamin preparation containing the PGA-vitamin complex of claim 1 as an effective ingredient.

10. Food containing the PGA-vitamin complex of claim 1 as an effective ingredient.

11. Beverage containing PGA-vitamin complex of claim 1 as an effective ingredient.

12. A cosmetic composition for improving skin compatibility, moisturizing property and/or hygroscopicity, containing PGA-vitamin complex of claim 1 as an effective ingredient.
